(19) United States
(12) Patent Application Publication
(10) Pub. No.: US 2021/0113360 A1
(43) Pub. Date: Apr. 22, 2021

(54) DEVICE FOR THE PREVENTION OF BEATING MOVEMENTS

(71) Applicant: Paul Gokkel, Utrecht (NL)

(72) Inventor: Gokkel Paul, Utrecht (NL)

(21) Appl. No.: 16/498,074

(22) PCT Filed: Apr. 9, 2018
(86) PCT No.: PCT/NL2018/050214

§ 371 Date: Sep. 26, 2019

(30) Foreign Application Priority Data
Apr. 9, 2017 (NL) 2018670

(51) Int. Cl. A61F 5/37; G08B 21/02; G08B 3/10

(52) U.S. Cl. CPC A61F 5/373; G08B 3/10; G08B 21/0286; A61F 5/3715

(57) ABSTRACT

The invention relates to a holder, which can be fixed around the elbows of a person to prevent that person to make hitting movements. By means of a belt, strap or a pin, which can be inserted through holes in both sides of said holder, or a shackle, the elbow can be secured in the holder. If the holder is fixed around someone's elbow, the wearer can not fully stretch his arm anymore, necessary for delivering a blow with his fist. However the arm still has sufficient freedom of movement and the user can still bend his arm to 90 degrees. Therefore the user can still perform all daily activities.

The present invention relates to a device to prevent a person to make hitting movements. By attaching a holder on one or both elbows or knees, the wearer can not effectively punch or kick somebody else. The holder is a user friendly solution for the major problem of aggression in healthcare, educational and correctional facilities. Contrary to fixation the user can perform all daily activities unhindered. Only fully stretching of the arms is blocked. In mentally handicapped care facilities, psychiatric hospitals, care homes, juvenile facilities and penitentiary institutions, staff members are frequently attacked. The greatest risk are punches by patients or patients throwing with objects. At present patients are often administered forced medication or they are isolated after an incident. This can be very frightening for the involved patients. Dit wordt door de betrokken patienten als erg bedreigend ervaren. Also anti-psychotic medication and tranquilizers are very harmful for the health of elderly with dementia. After an incident staff members and other patients are often very afraid for the aggressive patient. This impairs the treatment of that patient.

With the present invention isolation and compulsory medication can be prevented or reduced. If an aggressive patient is wearing the holder, he can stay with less risk in the common living area because he can not effectively fight anymore (This situation is comparable with a person wearing a sling or an arm in plaster) The patient will behave better and he is more amenable to treatment and behavioral interventions. Using the holder patients can return earlier and stay longer at home with informal caregiving. They pose then less risk to housemates, care workers and ambulatory nursing teams. With the holder the user is only blocked to punch or kick with stretched arms or legs. These (unanticipated) punches cause most injuries. There still remains the risk of an elbow strike, upper-cut and head-butt. However by preserving a certain distance (two feet) from the aggressor, one can avoid any attack.

An elbow strike, upper-cut and head-butt require a victim, staying much closer to the aggressor.

The invention relates to a holder that can be fixed around the elbows of the user. The holder comprises a central cavity having upstanding walls, which surrounds the elbow and leaving space for the arm at the top and front of said holder. The holder can comprise an aperture at its back and be embodied in that case as a open conical flexible sleeve. By means of a belt, strap or a pin, which can be inserted through holes in both sides of said holder, or a shackle, the elbow can be secured in the holder. The belt, strap, shackle or pin can be fastened to the holder by means of a (pad) lock. Because the aperture at the top of the holder is confined to a horizontal plane substantially above the upper rim of the aperture at the front of said holder, the wearer can not remove said holder if the belt, strap, shackle or pin is fastened to the holder by means of a lock. The holder can be made of leather, plastic or metal. This material must be not too flexible and must offer sufficient resistance to bending or stretching. The belt or strap can also be made from these materials. If the holder is tied around someone's elbow, the wearer can not fully stretch his arm anymore, needed for making a punching movement with his fist. However the arm still has sufficient freedom of movement and the user can still bend his arm to 90 degrees. Therefore the user can still perform all daily activities. The holder can also be mounted on the knees of a person to prevent kicking movements with the legs in the same way.

Another embodiment of the present invention comprises an integrated electronic alarm system, which makes an alarm when the user of said holder tries to remove said holder by cutting, sawing or breaking said holder by force.

The electronic detection system comprises a number, grid or array of electronic wires or cables in the interior of said holder and preferably in the interior of the belt strap or pin as well. In case of cutting, sawing or breaking open these parts by force, the power supply is then interrupted or changed, and the detection system generates an alarm signal. This alarm signal can produce an acoustic warning, or in case said holder includes a transmitter, sends an electronic signal to a distant receiver.

In the patent literature holders, which can be mounted on the elbow for protection, are described:

U.S. Pat. No. 4,953,569, Joint protector pad, R Lonardo, 1990 describes a protector pad, made of soft elastic material, which can be mounted on the elbow, for preventing skin abrasions at the joints of the user, when he bumps on his arm.

The present invention distinguishes itself from this prior art because it is made of rigid inelastic or little bendable material.

U.S. Pat. No. 6,273,091 Apparatus and method for safely maintaining a restraining hold on a person, B Chapman, 2001 relates to a hollow conical holder to restraint a person, lying on the ground, safely, pushing the elbows of said person into this holder and retaining the elbow with a belt. The present invention distinguishes itself from this patent application by comprising a detachable belt, such that elbow joint is located between the belt and the interior of the holder.

The present invention will be further elucidated here below with reference to the annexed figures of several examples of embodiments:

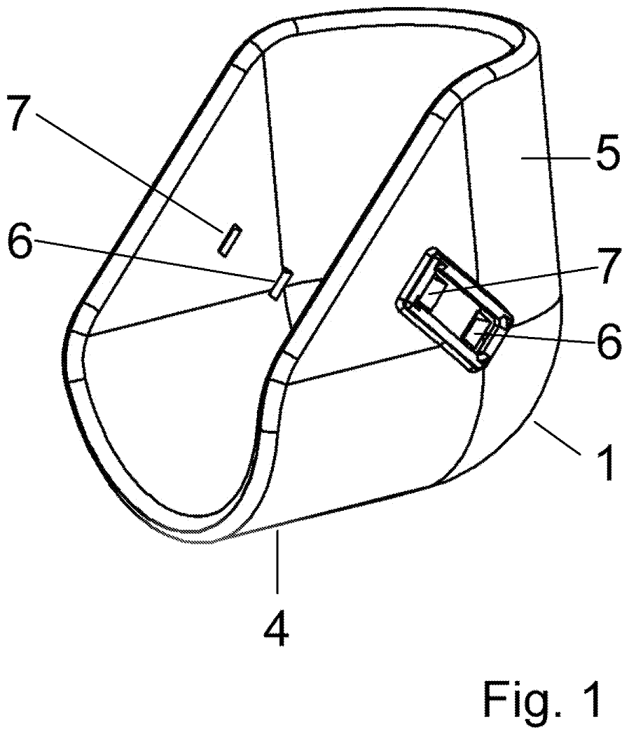

FIG. 1 shows the holder 1 comprising a horizontal hollow U-shaped member 4, in which the forearm can be inserted, and a vertical hollow U-shaped member 5, in which the upper arm can be embedded. The holder 1 includes in both side walls of the central part of said holder apertures 6 and 7, through which a belt, strap or tiewrap can be inserted to retain the wearer's arm at the elbow joint firmly into the holder 1.

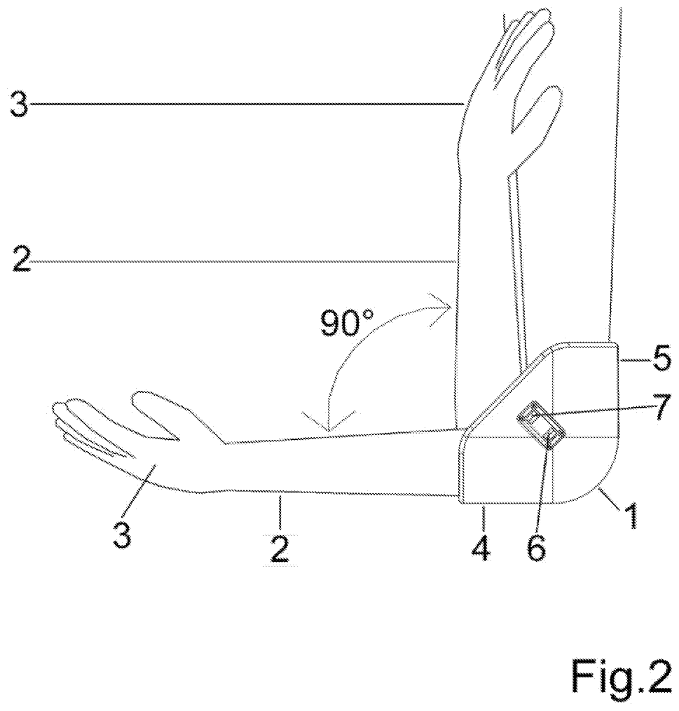

FIG. 2 depicts the operating principle of the holder 1. If the elbow is secured in the holder 1, the wearer of said holder 1 can not fully stretch his arm 2, which is needed to deliver a punch with the clenched fist 3. However the arm 2 still has enough freedom of movement and the user can bend his arm 2 unhindered to 90 degrees.

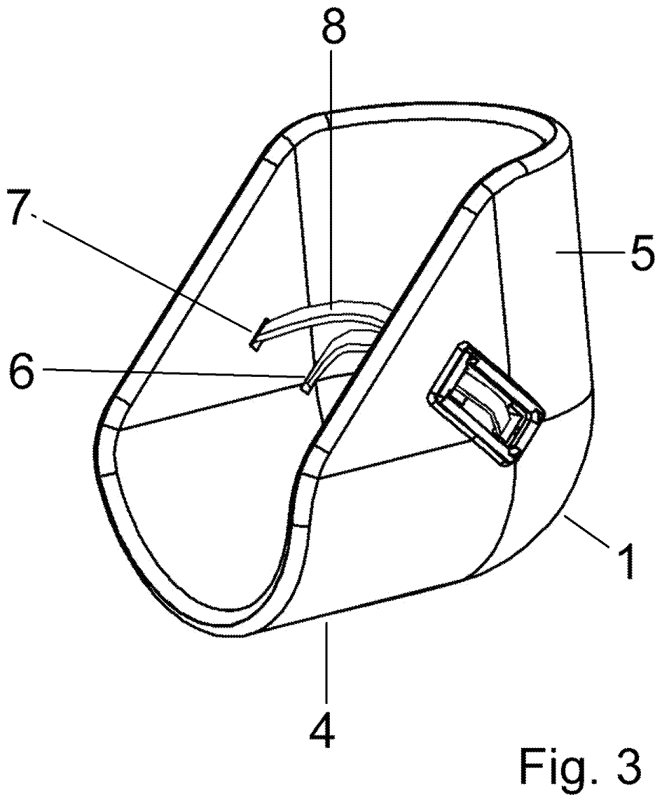

FIG. 3 shows a preferred embodiment of the holder 1 comprising a belt, strap, or tie wrap 8, which is inserted through both holes 6 and 7 in the side walls of the holder 1. This belt, strap, or tie wrap 8 fixes the elbow firmly in the holder 1, but providing the arm 2 enough freedom of movement to bend, so that the wearer can still perform all daily activities.

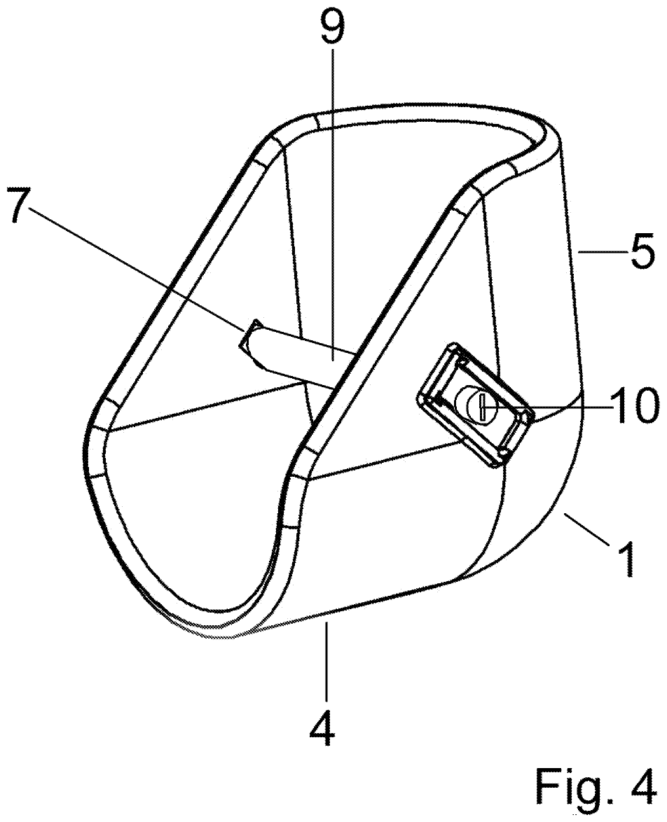

FIG. 4 depicts an embodiment of the holder 1, including a pin 9, which is inserted into the two holes 7 in the sidewalls of the holder 1. The pin 9 is fastened in the holder 1 by means of a lock 10. The wearer of the holder 1 can not remove the pin 9 without an appropriate key for the lock 10. The pin 9 fixes the user's elbow in the holder 1 and prevents the wearer of the holder 1 to pull his arm out of said holder 1 because the horizontally oriented hollow U-shaped member 4 of said holder 1 is bounded at its rear end by the vertical or skew oriented hollow U-shaped member 5 of said holder 1.

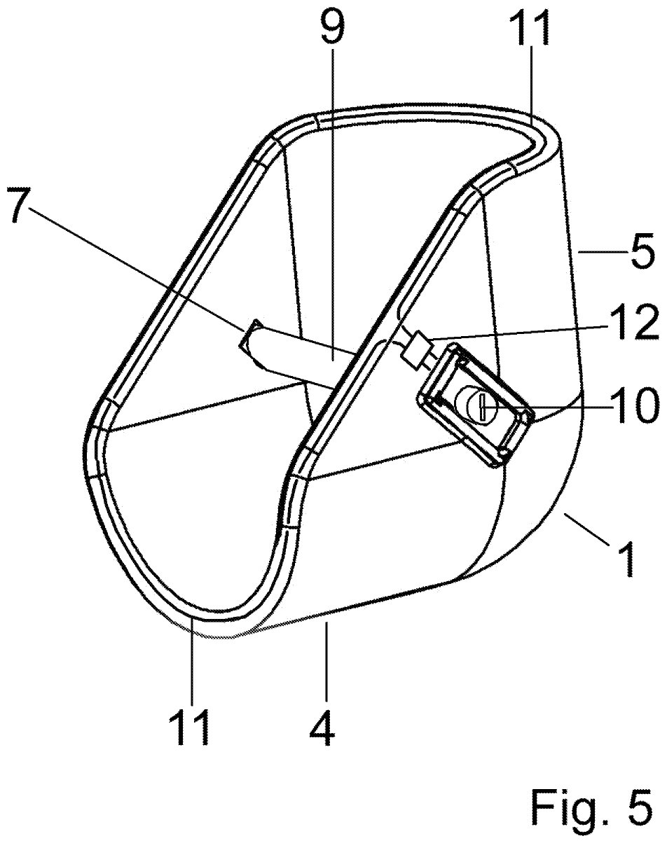

FIG. 5 shows the holder 1 comprising an integrated electronic detection system, which makes an alarm when the user of said holder 1 tries to remove said holder 1 by cutting or breaking said holder 1 by force. The electronic detection system comprises a number, grid or array of electronic wires or cables 11 in the interior of said holder 1 (In FIG. 5 only one cable 11 is depicted for clarity) and preferably in the interior of the belt, strap or pin 9 as well, such that in case of cutting, sawing or breaking open these parts by force, the power supply in these cables 11 is then interrupted or changed and an electronic control unit 12 in the detection system generates an alarm signal, producing an acoustic warning, or in case said holder 1 includes a transmitter, sends an electronic signal to a distant receiver.

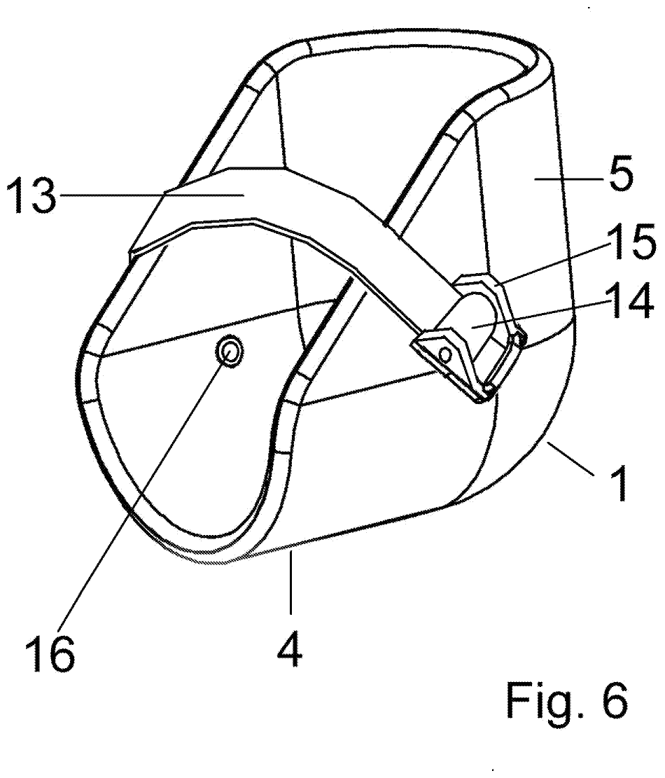

FIG. 6 depicts a preferred embodiment of the holder 1, including a shackle 13, which, by means of a hinge 14, is pivotally connected to an adaptor 15 of said holder 1. The shackle 13 is locked by means of a locking pin 16 to the opposite site wall of the holder 1 in a similar way as handcuffs. So one can apply the holder 1 more easily to reluctant users.

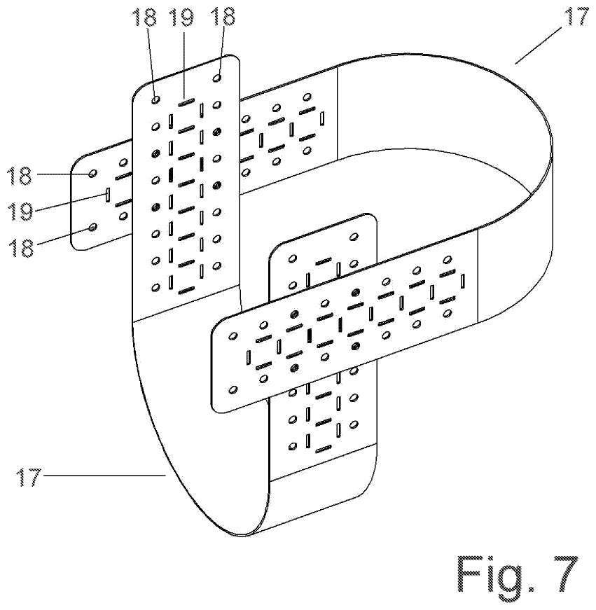

FIG. 7 shows the holder 1 comprising two rectangular strips 17, each of which are bent to an U-shape and with the ends attached to each other, perpendicularly oriented. The strips 17 include at both ends a regular array of holes 18 and 19. Both strips 17 are firmly secured together using blind rivets, bolts or tie wraps through these holes 18. The holes 18 of the perpendicularly attached strips 17 must therefore coincide in their area of attachment. The distance between both rows of holes 18 of one strip 17 must be equal to the distance between to corresponding holes 18 in one row of the other strip 17. Through the middle holes 19 a tie wrap or belt can be inserted analogous to the tie wrap or belt 8 in FIG. 3.

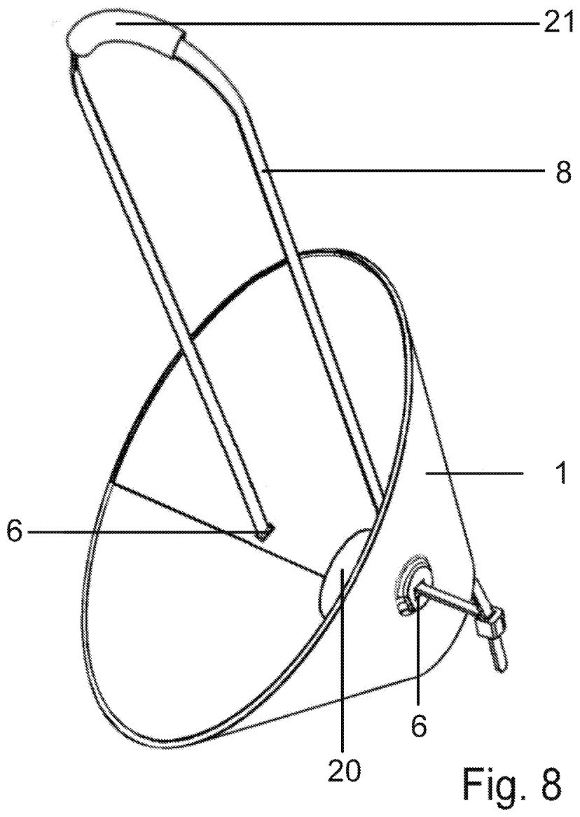

FIG. 8 depicts another preferred embodiment of the holder 1, comprising a conical holder 1, which includes at its rear end an aperture 20 and has at least two holes 6, through which a belt strap or tie wrap can be inserted to fixate the arm of the user at the elbow firmly in the holder 1. The tie wrap 8 is covered by a rubber hose 21 for more wearing comfort.

It will be apparent to the skilled person that the inventive concept allows a wide application and is certainly not limited to the given embodiments, which serve only by way of illustration.

1-10. (canceled)

11. Hollow holder which covers the elbow or knee of the wearer characterised by comprising a belt, strap, a shackle or pin, located substantially in the middle of the central part of the holder, residing near the inner side of the elbow or knee joint, to attach the holder around the elbow or knee and securely fasten said belt, strap or pin to said holder by means of a (pad)lock mechanism such that bending of the elbow of the wearer of said holder is limited substantially to the range of 0-90 degree angles, preventing the wearer to fully stretch his arm or leg for punching or kicking someone else and preventing the wearer to disconnect or remove said holder.

12. Hollow holder according to claim 11 characterised by comprising a shackle or metal link that is pivotally attached to one side of the holder and can be secured to the opposite side of the holder by means of a lock.

13. Hollow holder according to claim 11 characterised by comprising an elongated U profile, that is bent in its longitudinal direction so that one half of said U profile is horizontally oriented and includes the forearm or lower leg and the other half of the bent U profile is skew or vertically upwards oriented, including the upper arm or leg and the side walls in the curved center of the bent U profile comprise one or more attachment holes for inserting a belt, strap, tie wrap, shackle or pin.

14. Hollow holder according to claim 12 characterised by comprising an elongated U profile, that is bent in its longitudinal direction so that one half of said U profile is horizontally oriented and includes the forearm or lower leg and the other half of the bent U profile is skew or vertically upwards oriented, including the upper arm or leg, and the side walls in the curved center of the bent U profile comprise one or more attachment holes for inserting a shackle, a pin or metal link.

15. Hollow holder according to claim 13 whereby said holder comprises two perpendicularly oriented strips, each bent to an U shape, and each said strip includes at both its extremities, parallel to the long side of said strip a regular array of holes, with a fixed internal column distance.

16. Hollow holder according to claim 14 whereby said holder comprises two perpendicularly oriented strips, each bent to an U shape, and each said strip includes at both its extremities, parallel to the long side of said strip a regular array of holes, with a fixed internal column distance.

17. Hollow holder according to claim 13 characterised by comprising an (oval) cone shaped thin-walled surface, that leaves open a recess at the top of the cone and including at least two attachment holes in the sidewall, substantially at the middle of the cone for inserting a belt, strap, tie wrap, shackle or pin and said conical surface is substantially flexible to be pressed more ovally flat for precisely fitting different elbow or knee sizes.

18. Hollow holder according to claim 14 characterised by comprising an (oval) cone shaped thin-walled surface, that leaves open a recess at the top of the cone and including at least two attachment holes in the sidewall, substantially at the middle of the cone for inserting a shackle or pin and said conical surface is substantially flexible to be pressed more ovally flat for precisely fitting different elbow or knee sizes.

19. Hollow holder according to claim 13 characterised by comprising an integrated electronic detection system, which makes an alarm when the user of said holder tries to remove said holder by cutting or breaking said holder by force, said electronic detection system comprising a number, grid or array of electronic wires or cables in the interior of said holder and preferably in the interior of the belt strap or pin as well, such that in case of cutting, sawing or breaking open these parts by force, the power supply is then interrupted or changed and the detection system generates an alarm signal, producing an acoustic warning, or in case said holder includes a transmitter, sends an electronic signal to a distant receiver.

20. Hollow holder according to claim 14 characterised by comprising an integrated electronic detection system, which makes an alarm when the user of said holder tries to remove said holder by cutting or breaking said holder by force, said electronic detection system comprising a number, grid or array of electronic wires or cables in the interior of said holder and preferably in the interior of the belt strap or pin as well, such that in case of cutting, sawing or breaking open these parts by force, the power supply is then interrupted or changed and the detection system generates an alarm signal, producing an acoustic warning, or in case said holder includes a transmitter, sends an electronic signal to a distant receiver.

21. Hollow holder according to claim 15 characterised by comprising an integrated electronic detection system, which makes an alarm when the user of said holder tries to remove said holder by cutting or breaking said holder by force, said electronic detection system comprising a number, grid or array of electronic wires or cables in the interior of said holder and preferably in the interior of the belt strap or pin as well, such that in case of cutting, sawing or breaking open these parts by force, the power supply is then interrupted or changed and the detection system generates an alarm signal, producing an acoustic warning, or in case said holder includes a transmitter, sends an electronic signal to a distant receiver.

22. Hollow holder according to claim 16 characterised by comprising an integrated electronic detection system, which makes an alarm when the user of said holder tries to remove said holder by cutting or breaking said holder by force, said electronic detection system comprising a number, grid or array of electronic wires or cables in the interior of said holder and preferably in the interior of the belt strap or pin as well, such that in case of cutting, sawing or breaking open these parts by force, the power supply is then interrupted or changed and the detection system generates an alarm signal, producing an acoustic warning, or in case said holder includes a transmitter, sends an electronic signal to a distant receiver.

23. Hollow holder according to claim 17 characterised by comprising an integrated electronic detection system, which makes an alarm when the user of said holder tries to remove said holder by cutting or breaking said holder by force, said electronic detection system comprising a number, grid or array of electronic wires or cables in the interior of said holder and preferably in the interior of the belt strap or pin as well, such that in case of cutting, sawing or breaking open these parts by force, the power supply is then interrupted or changed and the detection system generates an alarm signal, producing an acoustic warning, or in case said holder includes a transmitter, sends an electronic signal to a distant receiver.

24. Hollow holder according to claim 18 characterised by comprising an integrated electronic detection system, which makes an alarm when the user of said holder tries to remove said holder by cutting or breaking said holder by force, said electronic detection system comprising a number, grid or array of electronic wires or cables in the interior of said holder and preferably in the interior of the belt strap or pin as well, such that in case of cutting, sawing or breaking open these parts by force, the power supply is then interrupted or changed and the detection system generates an alarm signal, producing an acoustic warning, or in case said holder includes a transmitter, sends an electronic signal to a distant receiver.
